(19) United States
(12) Patent Application Publication
(10) Pub. No.: US 2017/0367994 A1
(43) Pub. Date: Dec. 28, 2017

(54) TRANSDERMAL PREPARATION

(71) Applicant: KM Transderm Ltd., Osaka (JP)

(72) Inventors: Atsuyo HAMADA, Higashikagawa-shi (JP); Mitsuji AKAZAWA, Higashikagawa-shi (JP); Keiko YAMASAKI, Higashikagawa-shi (JP); Masaoki GOTO, Higashikagawa-shi (JP)

(73) Assignee: KM Transderm Ltd., Osaka (JP)

(21) Appl. No.: 15/700,405

(22) Filed: Sep. 11, 2017

Related U.S. Application Data
(63) Continuation of application No. 15/146,453, filed on May 4, 2016, now abandoned.
Application No. 15/146,453 is a continuation of application No. 14/343,146, filed on Jun. 23, 2014, now abandoned.

(30) Foreign Application Priority Data
Sep. 8, 2011 (JP) 2011-196062

(51) Int. Cl. A61K 9/70; A61K 31/44; A61K 31/445

(52) U.S. Cl. CPC A61K 9/7023; A61K 31/445; A61K 9/7053; A61K 31/44

(57) ABSTRACT

The present invention provides transdermal absorption preparation having a support, and a drug-containing adhesive layer formed on the support, wherein a drug-containing adhesive layer contains donepezil or a salt thereof, and a higher fatty acid salt.

### CROSS-REFERENCE TO RELATED APPLICATIONS

This patent application is a continuation of copending U.S. patent application Ser. No. 15/146,453, filed on May 4, 2016, which is a continuation of U.S. patent application Ser. No. 14/343,146, filed on Jun. 23, 2014, which is the U.S. national phase of International Patent Application No. PCT/JP2012/072933, filed Sep. 7, 2012, which claims the benefit of Japanese Patent Application No. 2011-196062, filed on Sep. 8, 2011, which are incorporated by reference in their entireties herein.

### TECHNICAL FIELD

The present invention relates to a transdermal absorption preparation containing donepezil or a salt thereof.

### BACKGROUND ART

Donepezil (i.e., 1-benzyl-4-(5,6-dimethoxyindanone-2-yl)methylpiperidine) is widely used for the treatment of mild to moderate levels of Alzheimer-type dementia, generally in the form of hydrochloride (i.e., donepezil hydrochloride). Alzheimer-type dementia is a disease wherein the normal function of the brain is gradually lost since the nerve cells constituting the brain decreases more rapidly than in general aging (denaturation). About 5% of the population of those aged 65 years or over are said to be dementia patients; 40% of which being Alzheimer-type, and the number of patients is the highest among the diseases associated with denaturation of nerve. The number of patients is expected to increase in the future aging society, and the treatment thereof will become more and more important. The action of donepezil on Alzheimer-type dementia is considered to be based on an increase in brain acetylcholine, which is achieved mainly by the inhibition of acetylcholinesterase and activation of the brain cholinergic nerve system.

Conventionally, donepezil is mainly administered orally, and has been marketed in the dosage form of tablet, jelly and the like. However, patients with worsened symptoms of dementia have difficulty in orally taking the medicine. Therefore, administration of donepezil by a pathway other than oral one, particularly, transdermal administration using a transdermal absorption preparation, has been desired.

As transdermal absorption preparations, tapes more superior in the adhesiveness are often used in recent years than poultices containing a large amount of water as a constituent component in the adhesive preparation. As an adhesive base for forming the adhesive layer of the tapes, lipophilic adhesive bases such as rubber-based adhesive base, acrylic adhesive base, silicone-based adhesive base and the like are used. Of these, rubber-based adhesive bases are widely used, since they are easily blended with additives as compared to other adhesive bases. However, a transdermal absorption preparation using a rubber-based adhesive base does not permit a sufficient release of the drug from the adhesive layer, and causes a problem of low transdermal absorbability of drugs such as donepezil and the like. While a tackifier is generally added to an adhesive base to confer or improve adhesiveness, a transdermal absorption preparation using a tackifier poses problems of skin irritation and the like due to the tackifier.

Patent document 1 describes use of an ester of fatty acid and lower alcohol such as diisopropyl adipate and the like to promote transdermal absorption of donepezil and the like in transdermal absorption preparations. Patent document 2 describes use of acetate to improve transdermal absorbability of donepezil hydrochloride in transdermal absorption preparations. However, the methods disclosed in these documents fail to provide good transdermal absorbability.

### DOCUMENT LIST

### Patent Documents

patent document 1: JP-A-H11-315016
patent document 2: WO 2003/032960

### SUMMARY OF THE INVENTION

### Problems to be Solved by the Invention

The present invention has been made taking note of the situation mentioned above, and aims to provide a transdermal absorption preparation showing good transdermal absorbability of donepezil.

### Means of Solving the Problems

The present inventors have conducted intensive studies in an attempt to solve the aforementioned problems and found that the transdermal absorbability of donepezil can be improved by adding higher fatty acid salt in addition to donepezil to an adhesive layer. The present invention based on such finding is as described below.
[1] A transdermal absorption preparation comprising a support, and a drug-containing adhesive layer formed on the support, wherein

the drug-containing adhesive layer comprises donepezil or a salt thereof, and a higher fatty acid salt.
[2] The transdermal absorption preparation of the above-mentioned [1], wherein the higher fatty acid has a carbon number of 12 or more and 30 or less.
[3] The transdermal absorption preparation of the above-mentioned [1] or [2], wherein the drug-containing adhesive layer comprises a higher fatty acid sodium.
[4] The transdermal absorption preparation of any one of the above-mentioned [1] to [3], wherein the drug-containing adhesive layer comprises a thermoplastic elastomer and liquid paraffin,

the content of the liquid paraffin in the drug-containing adhesive layer is more than 300 parts by weight and not more than 1500 parts by weight, per 100 parts by weight of the thermoplastic elastomer,

the content of the tackifier in the drug-containing adhesive layer is not more than 10 wt % (0 wt % inclusive).
[5] The transdermal absorption preparation of the above-mentioned [4], wherein the thermoplastic elastomer is a styrene-based block copolymer.
[6] The transdermal absorption preparation of the above-mentioned [5], wherein the styrene-based block copolymer is a styrene-isoprene-styrene block copolymer.
[7] The transdermal absorption preparation of any one of the above-mentioned [4] to [6], wherein the drug-containing adhesive layer does not contain a tackifier.
[8] The transdermal absorption preparation of any one of the above-mentioned [1] to [7], wherein the drug-containing adhesive layer contains an ester solvent and/or an alcohol solvent.
[9] The transdermal absorption preparation of any one of the above-mentioned [1] to [7], wherein the drug-containing adhesive layer contains an ester solvent and an alcohol solvent.

### Effect of the Invention

The transdermal absorption preparation of the present invention shows superior transdermal absorbability of donepezil.

### BRIEF DESCRIPTION OF THE DRAWINGS

FIG. 1 is a graph showing the transdermal permeation amount of donepezil 24 hr later in Experimental Example 1 (in vitro transdermal permeation test) using the transdermal absorption preparations of Examples 1-4 and Comparative Examples 1-3.

FIG. 2 is a graph showing the relationship between rat blood concentration of donepezil and adhesion time in Experimental Example 2 (in vivo transdermal permeation test) using the transdermal absorption preparation of Example 1.

### DESCRIPTION OF EMBODIMENTS

The transdermal absorption preparation of the present invention is a transdermal absorption preparation containing a support, and a drug-containing adhesive layer formed on the support, wherein the drug-containing adhesive layer contains donepezil or a salt thereof, and a higher fatty acid salt.

Donepezil refers to 1-benzyl-4-(5,6-dimethoxyindanone-2-yl)methylpiperidine. In the present invention, a pharmacologically acceptable donepezil salt can also be used. The drug-containing adhesive layer may contain both donepezil and a salt thereof. In addition, one kind of donepezil salt may be used, or two or more kinds thereof may be used in combination. From the aspects of easy availability and the like, donepezil or a salt thereof is preferably donepezil hydrochloride. The content of donepezil or a salt thereof (when they are use in combination, the total amount thereof) in the drug-containing adhesive layer is preferably 1-20 wt %, more preferably 2-15 wt %, to ensure dispersibility in a drug-containing adhesive layer and good transdermal absorbability, though the present invention is not limited thereto.

One of the characteristics of the present invention is use of a higher fatty acid salt to improve transdermal absorbability of donepezil. In the present invention, the “fatty acid” refers to, as described in RIKAGAKU JITEN (physics and chemistry dictionary) 5th Edition (IWANAMI SHOTEN), a chain monocarboxylic acid, the “higher fatty acid” refers to a fatty acid having a carbon number of not less than 10, and the “lower fatty acid” refers to a fatty acid having a carbon number of not more than 9.

The higher fatty acid may be a linear or branched chain. The higher fatty acid may be saturated or unsaturated. The carbon number of higher fatty acid is preferably not less than 12, more preferably not less than 14, further preferably not less than 16, and preferably not more than 30, more preferably not more than 24, further preferably not more than 20.

Examples of the saturated higher fatty acid include capric acid (carbon number 10), lauric acid (carbon number 12), myristic acid (carbon number 14), palmitic acid (carbon number 16), stearic acid (carbon number 18), isostearic acid (carbon number 18), arachidic acid (carbon number 20), behenic acid (carbon number 22), lignoceric acid (carbon number 24), cerotic acid (carbon number 26), montanic acid (carbon number 28), melissic acid (carbon number 30) and the like. Of these, myristic acid is preferable.

Examples of the unsaturated higher fatty acid include palmitoleic acid (carbon number 16), oleic acid (carbon number 18), linoleic acid (carbon number 18), (9,12,15)-linolenic acid (carbon number 18), (6,9,12)-linolenic acid (carbon number 18), eleostearic acid (carbon number 18) and the like. Of these, oleic acid and linoleic acid are preferable, and oleic acid is more preferable.

The higher fatty acid salt may be an inorganic salt or an organic salt. Examples of the inorganic salt include sodium salt, potassium salt, calcium salt and the like. As the organic salt, an amine salt and the like can be mentioned. From the aspects of easy availability, stability of donepezil in an adhesive layer and transdermal absorbability thereof and the like, the higher fatty acid salt is preferably an inorganic salt, more preferably a sodium salt.

The higher fatty acid salt is preferably at least one selected from the group consisting of oleate, myristate and linoleate, more preferably at least one selected from the group consisting of sodium oleate, sodium myristate and sodium linoleate, further preferably sodium oleate.

The content of the higher fatty acid salt in a drug-containing adhesive layer is preferably not less than 0.1 mol and more preferably not less than 0.2 mol, and preferably not more than 5 mol, more preferably not more than 3 mol, per 1 mol of donepezil, though the present invention is not limited thereto. When the amount of higher fatty acid salt is too small, a sufficient transdermal absorbability improving effect cannot be obtained. Conversely, when the amount of higher fatty acid salt is too high, the preparation property such as adhesive property and the like may be degraded.

In the transdermal absorption preparation of the present invention, the drug-containing adhesive layer contains a thermoplastic elastomer and liquid paraffin, the content of liquid paraffin in the drug-containing adhesive layer is preferably more than 300 parts by weight and not more than 1500 parts by weight, per 100 parts by weight of the thermoplastic elastomer, and the content of a tackifier in the drug-containing adhesive layer is preferably not more than 10 wt % (0 wt % inclusive). Using such a large amount of liquid paraffin, good adhesiveness can be achieved even when the content of a tackifier in the drug-containing adhesive layer is limited to 10 wt % or below. By limiting the content of the tackifier, a transdermal absorption preparation causing less skin irritation can be obtained. In addition, the presence of a large amount of liquid paraffin also improves transdermal absorbability of donepezil.

Only one kind of a thermoplastic elastomer may be used, or two or more kinds thereof may be used in combination. Here, the thermoplastic elastomer refers to a polymer showing flowability when heated, but showing properties similar to those of vulcanization rubber when cooled. The weight average molecular weight of the thermoplastic elastomer is preferably not less than 10,000, more preferably not less than 20,000, and preferably not more than 1,000,000, more preferably not more than 500,000. The weight average molecular weight can be measured by gel permeation chromatography.

The thermoplastic elastomer may be any of urethane-based thermoplastic elastomer, acrylic thermoplastic elastomer, styrene-based thermoplastic elastomer, olefin-based thermoplastic elastomer and the like. In view of the simultaneous achievement of good adhesiveness and low skin irritation, the thermoplastic elastomer is preferably a styrene-based thermoplastic elastomer, more preferably a styrene-based block copolymer. Examples of the styrene-based block copolymer include a styrene-butadiene block copolymer, a styrene-butadiene-styrene block copolymer, a styrene-isoprene block copolymer, a styrene-isoprene-styrene block copolymer, a styrene-ethylene/butylene block copolymer, a styrene-ethylene/butylene-styrene block copolymer, a styrene-ethylene/propylene block copolymer, a styrene-ethylene/propylene-styrene block copolymer, a styrene-isobutylene block copolymer, a styrene-isobutylene-styrene block copolymer and the like. In the aforementioned examples of the styrene-based block copolymer, a hyphen (−) is used to show the range of a block, and a slash (/) is used to show a copolymer block. For example, a “styrene-ethylene/butylene block copolymer” means a copolymer having a styrene block, an ethylene and a butylene copolymer block. Only one kind of a styrene-based block copolymer may be used or two or more kinds thereof may be used in combination.

Among the styrene-based block copolymers, a styrene-isoprene-styrene block copolymer, a styrene-isoprene block copolymer and a mixture of these are preferable, and a mixture of a styrene-isoprene-styrene block copolymer and a styrene-isoprene block copolymer is more preferable, from the aspects of simultaneous achievement of good adhesiveness and low skin irritation, availability and handling property.

The content of the styrene unit in the styrene-isoprene-styrene block copolymer is preferably 5-60 wt %, more preferably 10-50 wt %. The weight average molecular weight of the styrene-isoprene-styrene block copolymer is preferably not less than 20,000, more preferably not less than 30,000, and preferably not more than 500,000, more preferably not more than 300,000. The weight average molecular weight can be measured by gel permeation chromatography.

The content of the styrene unit in the styrene-isoprene block copolymer is preferably 5-50 wt %, more preferably 10-40 wt %. The weight average molecular weight of the styrene-isoprene block copolymer is preferably not less than 10,000, more preferably not less than 20,000, and preferably not more than 500,000, more preferably not more than 300,000. The weight average molecular weight can be measured by gel permeation chromatography.

In the present invention, the liquid paraffin refers to paraffin having a kinematic viscosity at 40° C. of 0.1-10000 cSt as measured according to ASTM D-445. The liquid paraffin is generally a mixture of alkane which is liquid at ambient temperature and has a carbon number of not less than 20. As the liquid paraffin, a commercially available product, particularly, one compatible with the pharmaceutical product-related standards defined in the Japanese Pharmacopoeia, the United States Pharmacopeia and the like can be preferably used. For example, liquid paraffin is commercially available from Sonneborn under the trade name of “KAYDOL”.

The content of the liquid paraffin in the drug-containing adhesive layer is preferably more than 300 parts by weight, more preferably not less than 320 parts by weight, and preferably not more than 1500 parts by weight, more preferably not more than 1000 parts by weight, per 100 parts by weight of the thermoplastic elastomer. When the amount of liquid paraffin is too small, good adhesiveness cannot be obtained. Conversely, when the amount of liquid paraffin is too high, the shape of the adhesive layer is difficult to maintain.

The content of the thermoplastic elastomer in the drug-containing adhesive layer is preferably not less than 5 wt %, more preferably not less than 8 wt %, further preferably not less than 10 wt %, and preferably not more than 23 wt %, more preferably not more than 22 wt %, further preferably not more than 20 wt %. When the amount of the thermoplastic elastomer is too small, the shape of the adhesive layer is difficult to maintain. Conversely, when the amount of the thermoplastic elastomer is too high, good adhesiveness cannot be obtained.

To reduce skin irritation, and the like, the content of the tackifier in the drug-containing adhesive layer is preferably not more than 10 wt %, more preferably not more than 5 wt %, further preferably not more than 2 wt %, particularly preferably not more than 1 wt %. It is most preferable that the drug-containing adhesive layer be free of a tackifier (i.e., tackifier content being 0 wt %).

Tackifier is well known in the field of adhesive preparations and generally means a resin used to confer adhesiveness to or improve adhesiveness of the adhesive base forming the adhesive layer. Examples of the tackifier include rosin resin, polyterpene resin, chroman-indene resin, petroleum resin, terpene-phenol resin, alicyclic saturated hydrocarbon resin and the like.

To further improve the transdermal absorbability of donepezil, the drug-containing adhesive layer preferably contains an ester solvent and/or an alcohol solvent. Here, only one kind of the ester solvent and the alcohol solvent may be used, or two or more kinds thereof may be used in combination.

Examples of the ester solvent include an ester of higher fatty acid and monovalent aliphatic alcohol, medium-chain triglyceride, an ester of polyvalent carboxylic acid and monovalent aliphatic alcohol, carbonate and the like.

Examples of the ester of higher fatty acid and monovalent aliphatic alcohol include myristates such as isopropyl myristate, ethyl myristate and the like, palmitates such as isopropyl palmitate, ethyl palmitate and the like, stearates such as isopropyl stearate and the like, oleates such as decyl oleate and the like, linoleates such as ethyl linoleate and the like, and the like. Examples of the medium-chain triglyceride include caprylic acid triglyceride, caproic acid triglyceride and the like. As fats and oils containing much medium-chain triglyceride, peanuts oil, olive oil, castor oil and the like can be mentioned. Examples of the ester of polyvalent carboxylic acid and monovalent aliphatic alcohol include sebacates such as diethyl sebacate, diisopropyl sebacate and the like, and adipates such as diethyl adipate, diisopropyl adipate and the like. Examples of the carbonate include propylene carbonate and the like. Of these, oleate, myristate, medium-chain triglyceride, sebacate, adipate, and carbonate are preferable, and diisopropyl adipate, decyl oleate, isopropyl myristate, medium-chain triglyceride, diethyl sebacate, and propylene carbonate are more preferable.

Examples of the alcohol solvent include higher alcohols such as benzyl alcohol, lauryl alcohol, isostearyl alcohol, 2-octyl dodecanol and the like; polyvalent alcohols such as ethylene glycol, glycerol, propylene glycol, 1,3-butanediol, polyethylene glycol having a molecular weight of about 100-600 and the like, and the like. Of these, polyvalent alcohols such as ethylene glycol, glycerol, propylene glycol, 1,3-butanediol, polyethylene glycol having a molecular weight of about 100-600 and the like are preferable, and ethylene glycol, propylene glycol, 1,3-butanediol, and polyethylene glycol having a molecular weight of about 100-600 are more preferable.

To enhance dispersibility of donepezil in a drug-containing adhesive layer and transdermal absorbability thereof, an ester solvent and an alcohol solvent are preferably used in combination. When they are used in combination, the weight ratio of ester solvent and alcohol solvent (i.e., ester solvent:alcohol solvent) is more preferably 1:1-1:4 to further improve the transdermal absorbability of donepezil.

The content of an ester solvent or an alcohol solvent (when they are use in combination, the total amount thereof) in the drug-containing adhesive layer is preferably not less than 3 wt %, more preferably not less than 5 wt %, and preferably not more than 50 wt %, more preferably not more than 40 wt %.

The transdermal absorption preparation has a support provided with a drug-containing adhesive layer. The support in the present invention is not particularly limited, and a support widely used in the art can be used. Examples of the support include stretchable or unstretchable woven fabric of polyethylene, polypropylene and the like; non-woven fabric; film of polyethylene, polypropylene, ethylene vinyl acetate copolymer, vinyl chloride, polyester (e.g., poly(ethylene terephthalate) (PET)) and the like; foamed supports of urethane, polyurethane and the like; and the like. The support may be a single layer structure or a laminate structure. Furthermore, to prevent accumulation of static electricity, an antistatic agent may be applied to the support. To achieve good anchor property with an adhesive layer, a non-woven fabric or woven fabric is preferably used as a support. The thickness of the support is preferably not less than 10 μm, more preferably not less than 15 μm, preferably not more than 100 μm, more preferably not more than 50 μm for a film, and preferably not less than 50 μm, more preferably not less than 100 μm, preferably not more than 2000 μm, more preferably not more than 1000 μm for a porous sheet such as woven fabric, non-woven fabric, foamed support and the like.

The transdermal absorption preparation may contain, as an optional component, surfactant, excipient, antioxidant, softening agent, flavor, colorant and the like. Only one kind of these optional components may be used, or two or more kinds thereof may be used in combination.

The surfactant may be any of anionic surfactant, nonionic surfactant, cationic surfactant and amphoteric surfactant. Examples of the surfactant include natural emulsifier, soap, polyoxyethylene sorbitan fatty acid ester, glycerine fatty acid ester, sorbitan fatty acid ester, polyoxyethylene higher alcohol ether, polyoxyethylene alkylphenol and the like.

Examples of the natural emulsifier include gum arabic, gelatin, tragacanth, lecithin, cholesterol and the like. Examples of the polyoxyethylene sorbitan fatty acid ester include monooleyl sorbitan polyoxyethylene and the like. Examples of the glycerine fatty acid ester include polyoxyethylene castor oil derivative, polyoxyethylene hydrogenated castor oil, glycerol monostearate and the like. Examples of the sorbitan fatty acid ester include sorbitan monostearate, sorbitan monolaurate, sorbitan monooleate, sorbitan sesquioleate, sorbitan trioleate and the like. As the polyoxyethylene higher alcohol ether, polyoxyethylene cetyl ether and the like can be mentioned. Examples of other surfactant include sodium alkyl sulfate (e.g., sodium lauryl sulfate etc.), polyoxyethylene polyoxypropylene copolymer (e.g., Pluronic etc.), cetyltrimethylammonium chloride and the like.

Examples of the excipient include silicon compound such as silicic anhydride, light anhydrous silicic acid, hydrous silicic acid and the like, cellulose derivative such as ethylcellulose, methylcellulose, hydroxypropylcellulose, hydroxypropylmethylcellulose and the like, water-soluble polymer such as polyvinyl alcohol and the like, aluminum compound such as dried aluminum hydroxide gel, hydrated aluminum silicate and the like, kaolin, titanium oxide and the like, and the like.

Examples of the antioxidant include dibutylhydroxytoluene, ascorbic acid, tocopherol, tocopherol ester derivative, butylhydroxyanisole, 2-mercaptobenzimidazole and the like.

The transdermal absorption preparation of the present invention can be produced by, for example, dissolving or dispersing an adhesive base containing donepezil and a higher fatty acid salt in a dilution solvent (for example, tetrahydrofuran) to prepare a coating solution of an adhesive base, applying the obtained coating solution of an adhesive base to a support, and then drying. The coating solution of an adhesive base can be applied and dried by a means well known in the field of adhesive preparations. The amount of the drug-containing adhesive layer after drying is preferably 10 g/m² or more, more preferably 20 g/m² or more, and preferably not more than 1000 g/m², more preferably not more than 800 g/m².

It is also possible to form a release liner on the drug-containing adhesive layer of the transdermal absorption preparation of the present invention. When a release liner is used, the transdermal absorption preparation can also be produced by applying the aforementioned coating solution of an adhesive base to a release liner and drying same to form a release liner provided with an adhesive layer, and laminating a support on the adhesive layer.

### EXAMPLE

The present invention is explained in more detail in the following by referring to Examples and Comparative Examples, which are not to be construed as limitative. In the following, “parts” and “%” mean “parts by weight” and “wt %”, unless specifically indicated otherwise.

### Examples 1-4 and Comparative Examples 1-3

A styrene-isoprene-styrene block copolymer (“JSR SIS5002”, manufactured by JSR) and liquid paraffin (“KAYDOL”, manufactured by Sonneborn) in the amounts described in the following Table 1 were dissolved in tetrahydrofuran (THF) to give a solution of a styrene-isoprene-styrene block copolymer and the like. Then, a fatty acid salt and donepezil hydrochloride in the amounts described in the following Table 1 were dissolved in an ester solvent and an alcohol solvent to give a solution of fatty acid salt and the like. The obtained solution of the styrene-isoprene-styrene block copolymer and the like and the solution of the fatty acid salt and the like were mixed to give a coating solution of an adhesive base. The obtained coating solution of the adhesive base was applied to a silicone-treated PET film (release liner) such that the amount of the drug-containing adhesive layer is 300 g/m² after drying. The release liner coated with the adhesive base was dried in an oven at 80° C. for 30 min, and a PET film (support) was laminated on the surface of the obtained adhesive layer to give a laminated sheet. The laminated sheet was cut into a desired size to give the transdermal absorption preparations of Examples 1-4 and Comparative Examples 1-3. The transdermal absorption preparation of Comparative Example 3 using acetate showed crystallization and drug formulation was a failure.

TABLE 1

| | | | | | | Comp. | Comp. | Comp. |
|---|---|---|---|---|---|---|---|---|
| | | Ex. 1 | Ex. 2 | Ex. 3 | Ex. 4 | Ex. 1 | Ex. 2 | Ex. 3 |
| styrene-isoprene- | | 10.0 | 7.5 | 10.0 | 10.0 | 10.0 | 10.0 | 10.0 |
| styrene block | | | | | | | | |
| copolymer (parts) | | | | | | | | |
| liquid paraffin | | 63.0 | 52.0 | 65.0 | 64.4 | 68.0 | 63.0 | 67.0 |
| (parts) | | | | | | | | |
| fatty acid | sodium | 5.0 | 7.5 | — | — | — | — | — |
| salt (parts) | oleate | | | | | | | |
| | sodium | — | — | — | — | — | — | 1.0 |
| | acetate | | | | | | | |
| | sodium | — | — | 3.0 | — | — | — | — |
| | myristate | | | | | | | |
| | sodium | — | — | — | 3.6 | — | — | — |
| | linoleate | | | | | | | |
| ester solvent | diisopropyl | 5.0 | 7.5 | 5.0 | — | 5.0 | 10.0 | 5.0 |
| (parts) | adipate | | | | | | | |
| | ethyl | — | — | — | 5.0 | — | — | — |
| | linoleate | | | | | | | |
| alcohol | propylene | 12.0 | 18.0 | 12.0 | 12.0 | 12.0 | 12.0 | 12.0 |
| solvent | glycol | | | | | | | |
| (parts) | | | | | | | | |

| | | | | | | | |
|---|---|---|---|---|---|---|---|
| donepezil | 5.0 | 7.5 | 5.0 | 5.0 | 5.0 | 5.0 | 5.0 |
| hydrochloride (parts) | | | | | | | |
| total of above | 100.0 | 100.0 | 100.0 | 100.0 | 100.0 | 100.0 | 100.0 |
| components (parts) | | | | | | | |
| amount of fatty acid | 1.4 | 1.4 | 1.0 | 1.0 | — | — | 1.0 |
| salt (mol) per 1 mol | | | | | | | |
| of donepezil | | | | | | | |
| hydrochloride | | | | | | | |
| THF (parts) | 100.0 | 100.0 | 100.0 | 100.0 | 100.0 | 100.0 | 100.0 |

### Experimental Example 1

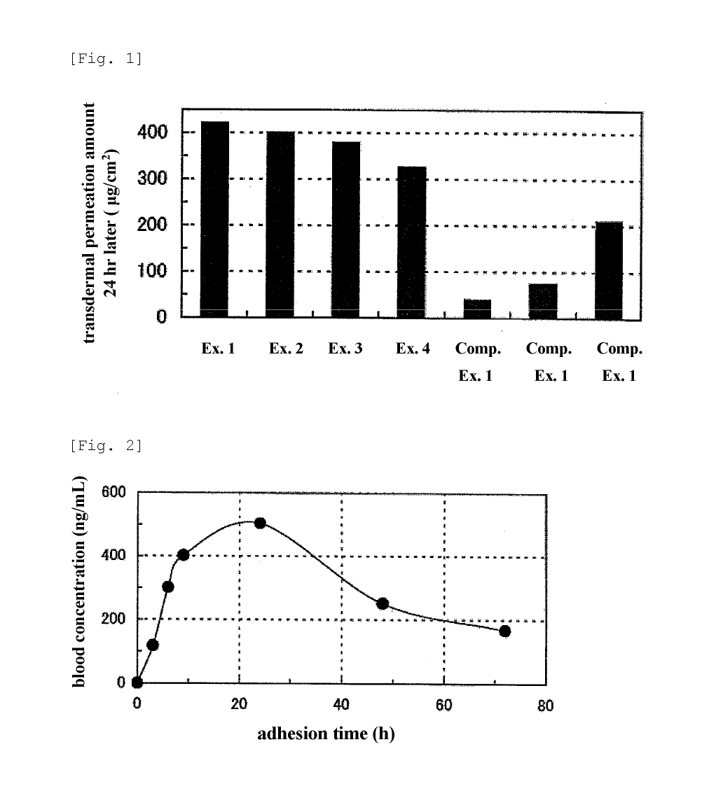

Using the transdermal absorption preparations of Examples 1-4 and Comparative Examples 1-3, the following in vitro transdermal permeation test was performed. An abdominal skin of male Wister rat (5-week-old) was set on a vertical Franz diffusion cell. Then, the transdermal absorption preparation was cut out in a circular shape with diameter 1.0 cm, the release liner was detached and the preparation was adhered to the rat skin on the diffusion cell (n=3). In the receptor side, the amount of donepezil that permeated through the rat skin after a given time was measured by high performance liquid chromatography (HPLC) using an ethanol-saline mixed solution (ethanol amount: 10%). The measurement results (permeation amount of donepezil 24 hr after adhesion) are shown in FIG. 1. The measurement conditions of HPLC are shown below:

### <HPLC Conditions>

HPLC system: high performance liquid chromatogram (LC2010C) manufactured by SHIMADZU CORPORATION

column: ODS, 4.6 mmφ×15 cm, 5 μm

column temperature: 40° C.

mobile phase: 0.1% aqueous phosphoric acid solution/methanol/acetonitrile/SDS=4/1/5/0.01 (weight ratio)

detection wavelength: 271 nm

flow: 0.7 mL/min

From FIG. 1, it is clear that the transdermal absorption preparation of the present invention shows high transdermal absorbability of donepezil. To be specific, the transdermal absorption preparations of Examples 1-4 using a higher fatty acid salt showed high transdermal permeation amounts of donepezil as compared to the transdermal absorption preparation of Comparative Example 2 using a high amount of diisopropyl adipate, which is described in patent document 1, and the transdermal absorption preparation of Comparative Example 3 using acetate, which is described in patent document 2.

In the transdermal absorption preparation of Comparative Example 3 using a lower fatty acid salt (acetate), crystal was precipitated from a drug-containing adhesive layer, and drug formulation was a failure. Such crystallization was not seen in the transdermal absorption preparations of Examples 1-4 using a higher fatty acid salt. It also demonstrates that a higher fatty acid salt is advantageously used for a donepezil-containing transdermal absorption preparation.

### Experimental Example 2

Using the transdermal absorption preparation of Example 1, the following in vivo transdermal permeation test was performed. The transdermal absorption preparation of Example 1 was cut into 4 cm×6 cm, the release liner was detached and the preparation was adhered to the back of five male slc/HWY hairless rats (7-week-old). Immediately after adhesion (0 hr later) and 3, 6, 9, 24, 48 and 72 hr later, blood samples were collected from the cervical vein of the rat, and the collected blood was centrifuged to obtain plasma. The concentration of donepezil in the obtained plasma was measured by liquid chromatography-tandem mass spectrometry (LC-MS/MS). The measurement results are shown in FIG. 2. The measurement conditions of LC-MS/MS are shown below:

### <LC Conditions>

HPLC system: high performance liquid chromatogram (1200 Series) manufactured by Agilent Technologies

analysis column: Atlantis dc18, 2.1 mm I.D.×150 mm, 5 μm

column temperature: 40° C.

mobile phase: methanol/0.05% formic acid solution

flow: 0.2 mL/min

### <MS/MS Conditions>

Tandem mass spectrometer: API4000, manufactured by AB Sciex Pte

interface: Turbo-V spray

ionization method: ESI, plus ion mode

measurement ion: 380.5 (donepezil), 91.1 (fragment ion)

From FIG. 2, it is clear that donepezil is transdermally absorbed from the transdermal absorption preparation obtained in Example 1, and transferred into blood.

### Experimental Example 3

Using the transdermal absorption preparation of Example 3, the following primary skin irritation test was performed. The transdermal absorption preparation of Example 3 was cut into 2.5 cm×2.5 cm, the release liner was detached and the preparation was adhered to the back, from which the hair had been removed with an electric clipper, of male white rabbit (Kbs:NZW). After occlusive adhesion for 24 hr, the transdermal absorption preparation was detached and, at 1, 24, 48 and 72 hr from the detachment, the primary irritation index value P.I.I. value was calculated by the method of Draize. J.H. et al. described in J. Pharmacol. Exp. Ther. 82: 377-390, and skin irritation was evaluated. The calculated P.I.I. value was 1.4, and the transdermal absorption preparation of Example 3 was evaluated to be weakly irritating. From the results, it is clear that the transdermal absorption preparation of the present invention shows low skin irritation.

### INDUSTRIAL APPLICABILITY

The transdermal absorption preparation of the present invention shows good transdermal absorbability of donepezil, and is useful as an agent for Alzheimer-type dementia.

This application is based on a patent application No. 2011-196062 filed in Japan, the contents of which are incorporated in full herein.

1. A transdermal absorption preparation comprising a support, and a drug-containing adhesive layer formed on the support, wherein
the drug-containing adhesive layer comprises donepezil or a salt thereof, and a higher fatty acid salt, and
the higher fatty acid salt consists of a salt of a higher fatty acid containing a carbon number of 12 or more and 30 or less.

2. The transdermal absorption preparation according to claim 1, wherein the drug-containing adhesive layer comprises a sodium salt of a higher fatty acid sodium.

3. The transdermal absorption preparation according to claim 1, wherein
the drug-containing adhesive layer comprises a thermoplastic elastomer and liquid paraffin,
the content of the liquid paraffin in the drug-containing adhesive layer is more than 300 parts by weight and not more than 1500 parts by weight, per 100 parts by weight of the thermoplastic elastomer, and
the content of the tackifier in the drug-containing adhesive layer is from 0 wt % to not more than 10 wt %.

4. The transdermal absorption preparation according to claim 3, wherein the thermoplastic elastomer is a styrene-based block copolymer.

5. The transdermal absorption preparation according to claim 4, wherein the styrene-based block copolymer is a styrene-isoprene-styrene block copolymer.

6. The transdermal absorption preparation according to claim 3, wherein the drug-containing adhesive layer does not contain a tackifier.

7. The transdermal absorption preparation according to claim 6, wherein the drug-containing adhesive layer contains an ester solvent and an alcohol solvent.

8. The transdermal absorption preparation according to claim 1, wherein the drug-containing adhesive layer contains an ester solvent and/or an alcohol solvent.

9. The transdermal absorption preparation according to claim 1, wherein the drug-containing adhesive layer contains an ester solvent and an alcohol solvent.

10. The transdermal absorption preparation according to claim 9, wherein the drug-containing adhesive layer comprises a sodium salt of a higher fatty acid sodium.

11. The transdermal absorption preparation according to claim 2, wherein the drug-containing adhesive layer comprises a thermoplastic elastomer and liquid paraffin,
the content of the liquid paraffin in the drug-containing adhesive layer is more than 300 parts by weight and not more than 1500 parts by weight, per 100 parts by weight of the thermoplastic elastomer, and
the content of the tackifier in the drug-containing adhesive layer is from 0 wt % to not more than 10 wt %.

12. The transdermal absorption preparation according to claim 11, wherein the thermoplastic elastomer is a styrene-based block copolymer.

13. The transdermal absorption preparation according to claim 12, wherein the styrene-based block copolymer is a styrene-isoprene-styrene block copolymer.

14. The transdermal absorption preparation according to claim 13, wherein the drug-containing adhesive layer does not contain a tackifier.

15. The transdermal absorption preparation according to claim 14, wherein the drug-containing adhesive layer contains an ester solvent and/or an alcohol solvent.

16. The transdermal absorption preparation according to claim 14, wherein the drug-containing adhesive layer contains an ester solvent and an alcohol solvent.

17. The transdermal absorption preparation according to claim 1, wherein the content of the higher fatty acid salt in the drug-containing adhesive layer is not less than 0.1 mol and not more than 5 mol per 1 mol of donepezil.
